(19) United States
(12) Patent Application Publication
(10) Pub. No.: US 2026/0347329 A1
(43) Pub. Date: Nov. 5, 2026

(54) NOVEL SURFACTANT COMPOUNDS

(71) Applicant: NORTH-WEST UNIVERSITY, Potchefstroom (ZA)

(72) Inventors: Desmond Austin YOUNG, Vanderbijlpark (ZA); Hermanus Cornelius Moolman VOSLOO, Potchefstroom (ZA); Frans Thomas Ignatius MARX, Harbeespoort (ZA); David VENTER, Potchefstroom (ZA)

(21) Appl. No.: 19/486,594

(22) PCT Filed: May 24, 2024
(86) PCT No.: PCT/IB2024/055076

§ 371 Date: Nov. 21, 2025

(30) Foreign Application Priority Data
May 26, 2023 (ZA) 2023/05668
Feb. 27, 2024 (ZA) 2024/01654

(51) Int. Cl. C07D 265/33; C07C 21/04; C07C 21/073; C07C 33/025; C07C 33/035; C07C 229/08; C07C 229/12; C07C 229/16; C07C 229/24; C07C 229/60

(52) U.S. Cl. CPC C07D 265/33; C07C 21/04; C07C 21/073; C07C 33/025; C07C 33/035; C07C 229/08; C07C 229/12; C07C 229/16; C07C 229/24; C07C 229/60

(57) ABSTRACT

The present invention relates to novel surfactant compounds. Particularly, novel types of Guerbet-type surfactant compounds which have been shown to demonstrate superior surfactant properties.

### FIELD OF APPLICATION OF THE INVENTION

The present invention relates to novel surfactant compounds. Particularly, novel types of Guerbet-type surfactant compounds.

### BACKGROUND TO THE INVENTION

Surfactants are molecules which possess both hydrophobic and hydrophilic groups. This characteristic allows the surfactant to bind with oil and water simultaneously, thereby lowering the surface tension of water as example. A wide range of synthetic surfactants exist today and can be applied for different purposes.

The four broad categories into which surfactants can be classified include amphoteric (or zwitterionic), anionic, cationic, and non-ionic.

Zwitterionic surfactants, which contain both positively and negatively charged head groups, are interesting molecules because of their many unique properties. In general, they are mild to the skin and eyes, exhibit low toxicity, display excellent water solubility, broad isoelectric ranges, high foam stability, resistance to hard water, and degradation by oxidizing agents. Factors affecting zwitterionic surfactants to various degrees may include but are not limited to, temperature, pH, and added electrolytes.

Cationic surfactants are surfactants that have a positively charged functional group (hydrophilic head) whilst anionic surfactants are surfactants that carry a negatively charged functional group (hydrophilic head).

Guerbet alcohols are a type of compound named after the French chemist Marcel Guerbet, who first discovered them in 1899. Guerbet was investigating the reactions of alcohols with sodium metal when he discovered a new type of alcohol with a branched structure. He later found that these branched alcohols could be used as surfactants.

In the early 20th century, Guerbet alcohols were used mainly as solvents and intermediates in the synthesis of other compounds. It was not until the 1950s that their surfactant properties were fully realized, and they began to be used in a range of industrial applications. Guerbet-type surfactants became increasingly popular in the 1970s and 1980s as more industries discovered their unique properties. The branched structure and unique functional groups of Guerbet-type surfactants give them excellent surface activity and stability, making them highly effective in reducing surface tension and stabilizing emulsions.

The popularity of Guerbet-type surfactants continued to grow in the 1990s and 2000s as advances in technology and chemistry allowed for the development of new Guerbet-type surfactant formulations with improved properties.

Due to their versatile properties, they are utilized in various industries which include but are not limited to, detergents, personal care, pharmaceuticals, agrochemicals, mining, metalworking, paper, lubricating products, latex systems, plastics, and composites.

Regarding the surfactants market size, in the year 2020, the global surfactants market was valued at USD 33 billion and projected to grow at a CAGR of 3.5% between 2021-2027, reaching the USD 44 billion mark by the end of 2027. Of interest, Guerbet-type surfactants are expected to grow from USD 1.1 billion in 2021 to USD 1.4 billion in 2029 at a CAGR of 3.9%. The cosmetics and personal care segment is projected to be the main driver of the global Guerbet alcohols market during the forecast period, with the Asia-Pacific region being the market leader. In this period, it is projected that the anionic amino acid surfactants will lose market share to cationic and zwitterionic surfactants.

The unique properties of Guerbet-type surfactants make them valuable and versatile in many industries, with the potential for further expansion as research and technology evolve. However, Guerbet-type surfactants have a unique chemical structure consisting of branched alkyl chains that can make them more challenging to manufacture compared to other surfactants with linear alkyl chains. This structural complexity requires specialised production processes and equipment, which can result in higher production costs and lower yields. As a result, the supply of Guerbet-type surfactants may be limited, and they may not be as widely available as other types of surfactants.

The limited availability of Guerbet-type surfactants can also make them more expensive to obtain. The higher product costs associated with manufacturing these surfactants can translate into higher prices for consumers. Additionally, because they may not be produced in as large quantities as other surfactants, suppliers may need to charge a premium for Guerbet-type surfactants to cover their expenses and maintain profitability.

The limited availability of Guerbet-type surfactants can also create logistical challenges for companies that rely on these surfactants in their products. Companies may need to establish relationships with specialised suppliers or invest in their own production capabilities to ensure a consistent supply of Guerbet-type surfactants. In addition, hereto, some Guerbet-type surfactants can only be obtained under licence from patentees which, evidently, can be very expensive. These added costs and challenges can make it harder for some companies to use Guerbet-type surfactants and may limit their use in niche applications or high-end products.

Although there is a growing trend toward the demand for bio-based surfactants, the high cost of manufacturing these surfactants has a negative impact on the forecasted growth of this market. Bio-surfactants are more expensive in comparison to traditional synthetic surfactants. The high costs of producing bio-surfactants are driven by the high cost of raw materials and offset economies of scale of bio-surfactants. This is particularly true in the case of fast-moving consumer goods (FMCG) companies such as Reckitt Benckiser, P&G, L'Oréal, and Colgate where the raw material prices have a definite impact on the consumer goods. For these reasons, there is a huge demand for synthetic surfactants.

Given the above, the unique chemical structure and specialised production processes associated with Guerbet-type surfactants can make them more expensive and harder to obtain than other types of surfactants, which can limit their availability and use in certain applications. There thus exists a clear need in the art for synthesised novel surfactants, particularly, novel Guerbet-type surfactants, which can be manufactured less expensively.

### OBJECT OF THE INVENTION

Accordingly, it is an object of the present invention to provide novel surfactant compounds, particularly novel Guerbet-type surfactant compounds, which overcome, at least partially, the abovementioned disadvantages and limitations and/or which will provide a useful alternative to existing technology mitigating the high cost of manufacturing the currently used Guerbet-type surfactants.

### SUMMARY OF THE INVENTION

According to a first aspect of the present invention, there is provided for novel surfactant compounds having the chemical structure:

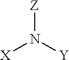

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) Y is selected from the group consisting of H; R′, X; (CH₂)ₙCO₂M (n=0, 1, 2, . . . ; M=H, R′, Li, Na, K, Rb, Cs, Fr, Er, Gd, . . . ); (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)); or CH₂CH(CH₃)O(CH₂CH₂O)ₙH (n=0, 1, 2, . . . ); and
(iii) Z is selected from the group consisting of H; R′; (CH₂)ₙCO₂M (n=1, 2, . . . ; M=H, R′, Li, Na, K, Rb, Cs, Fr, Er, Gd, . . . ); CH₂CH(CH₃)O(CH₂CH₂O)ₙH (n=0, 1, 2, . . . ); or (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)).

According to a second aspect of the present invention, there is provided for novel surfactant compounds having the chemical structure:

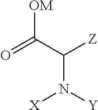

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) Y is selected from the group consisting of H; R′, X; (CH₂)ₙCO₂M (n=0, 1, 2, . . . ; M=H, R′, Li, Na, K, Rb, Cs, Fr, Er, Gd, . . . ); (CH₂)ₙA (n=0, 1, 2, . . . ; A=OH, OR′, SH, SR′, NH₂, NHR′, NR′₂, CONH₂, CONHR′, CONR′₂, aromatic groups, heteroaromatic groups, halogens (F, Cl, Br, I)); or CH₂CH(CH₃)O(CH₂CH₂O)ₙH (n=0, 1, 2, . . . ); and
(iii) Z is selected from the group consisting of H; R′; (CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); (CH₂)ₙCO₂M (n=1, 2, . . . ; M=H, R′, Li, Na, K, Rb, Cs, Fr, Er, Gd, . . . );

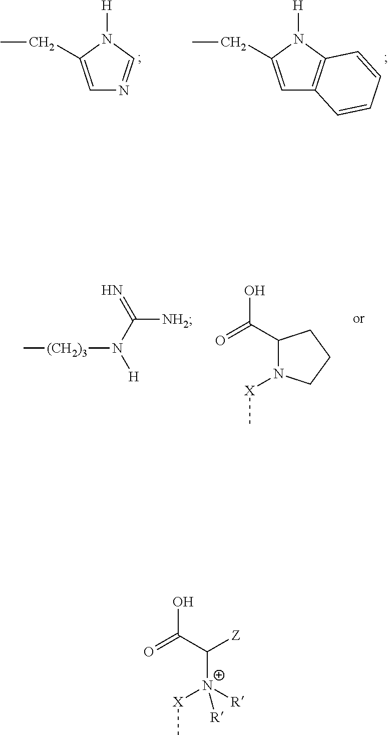

wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine.

According to a third aspect of the present invention, there is provided for novel surfactant compounds having the chemical structure:

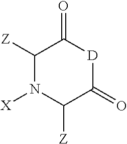

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) D is selected from the group consisting of O; N(CH₂)ₙA or N(C₆H₄)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups or halogens (F, Cl, Br, I)); and
(iii) Z is selected from the group consisting of H; R′; N(CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); or (CH₂)ₙCO₂M (n=1, 2, . . . ; M=H, R′, Li, Na, K, Rb, Cs, Fr, Er, Gd, . . . ).

According to a fourth aspect of the present invention, there is provided for novel surfactant compounds having the chemical structure:

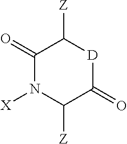

wherein:
(i) X is either COR or CH₂R, wherein R is either R′CH(CH₂)₂R′ or R′CCHCH₂R′, and wherein R′ is CₙH₂ₙ₊₁ (n=1, 2, . . . );
(ii) D is selected from the group consisting of O; N(CH₂)ₙA or N(C₆H₄)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups or halogens (F, Cl, Br, I)); and
(iii) Z is selected from the group consisting of H; R′; N(CH₂)ₙA (n=0, 1, 2, . . . ; A=OH, OR′, SH, SR′, NH₂, NHR′, NR′₂, CONH₂, CONHR′, CONR′₂, aromatic groups (e.g. C₆H₅, C₆H₄OH), heteroaromatic groups, halogens (F, Cl, Br, I)); or (CH₂)ₙCO₂M (n=1, 2, . . . ; M=H, R′, Li, Na, K, Rb, Cs, Fr, Er, Gd, . . . ).

The above and other characteristics, features, and advantages of the present invention will become apparent from the following detailed description, which illustrates, by way of example, the principles of the invention. This description is given for the sake of an example only, without limiting the scope of the invention.

### DESCRIPTION OF PREFERRED EMBODIMENTS OF THE INVENTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

A non-limiting example of preferred embodiments of the invention is described in more detail below.

### Example 1

The surfactant compounds in accordance with the present invention can be synthesized in terms of the following synthesis routes.
AC=aldol condensation
AH=acid hydrolysis
Am=amidation
[dH]=dehydrogenation
E=esterification (M=R)
G=Guerbet reaction
HAM=hydroaminomethylation
HC=hydrocyanation
HF=hydroformylation
[H]=hydrogenation
MC=methoxycarbonylation
[O]=oxidation
OM=olefin (alkene) metathesis
(i)=reductive amination
(ii)=amidation via acid halide
D=O, NH₍₁₋ₓ₎Rₓ
M=metal, alkyl
R, R′, R″=alkyl, etc.
X═CH₂ (amine), C═O (amide)

The source of olefins (alkenes) can be industrial (i.e. Fischer-Tropsch synthesis, oligomerization of ethene, alkane (paraffin) cracking, hydrogenation of alkynes, etc.) or natural (i.e. oleochemicals, sugars, etc.).

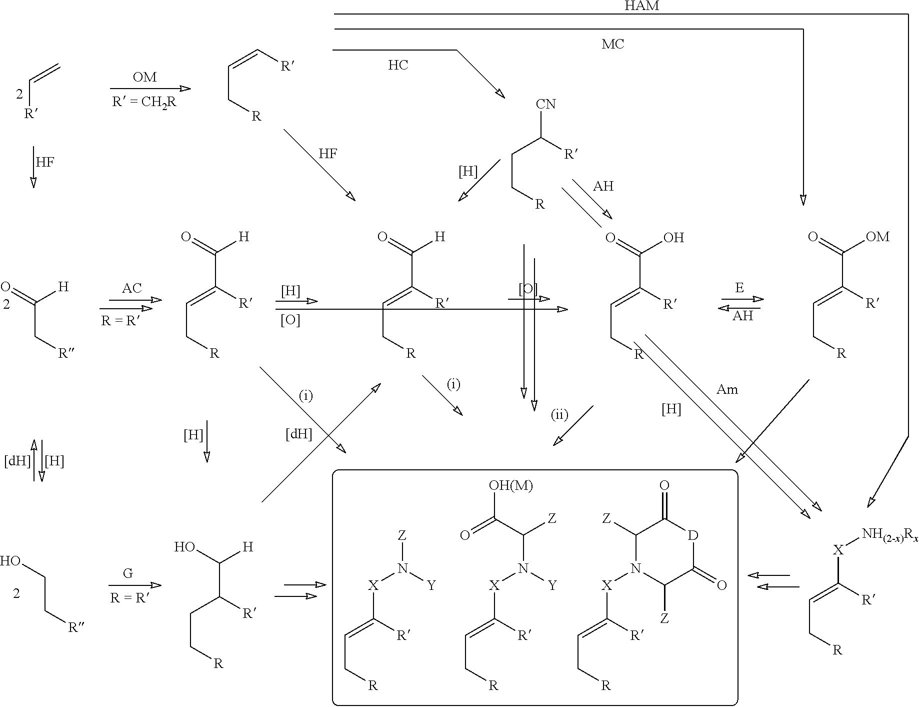

The surfactant compounds in accordance with the first aspect of the present invention, as synthesized from the above synthesis route, are shown below in Table 1.

TABLE 1
Surfactant compounds in accordance with the first aspect of the present invention

| | | | Synthesised and |
|---|---|---|---|
| | Y | Z | characterised |
| 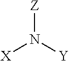 | H H H | H R′ (CH₂)ₙA | n = 0: bCₙp-ABA n = 2, A = OH,Cl: bCₙmEA, |
| | | | bCₙmEC |
| | R′ or X | (CH₂)ₙA | |
| | R′ or X | (CH₂)ₙCO₂M | |
| | R′ or X | CH₂CH(CH₃)O(CH₂CH₂O)ₙH | |
| | (CH₂)ₙA | (CH₂)ₙA | n = 2, A = OH,Cl: |
| | | | bCₙdEA, bCₙdEC |
| | (CH₂)ₙCO₂M | (CH₂)ₙCO₂M | |
| | CH₂CH(CH₃)O(CH₂CH₂O)ₙH | CH₂CH(CH₃)O(CH₂CH₂O)ₙH | |

X = COR or CH₂R
R = R′CH(CH₂)₂R′ or R′CCHCH₂R′
R′ = alkyl (CₙH₂ₙ₊₁; n = 1, 2, . . .)
n = 0, 1, 2, . . .
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd
bCₙ = R(CH₂)₂CHRCH₂—;
bCₙ(O) = R(CH₂)₂CHRC(═O)—
beCn = R(CH₂)₂CHRCH₂—;
beCn(O) = R(CH₂)₂CHRC(═O)—
p-ABA = para-aminobenzoic acid,
mEA = monoethylenealcohol,
dEA = diethylenealcohol,
mEC = monoethylenechloride,
dEC = diethylenechloride

The surfactant compounds in accordance with the second aspect of the present invention, as synthesized from the above synthesis route, are shown below in Table 2.

TABLE 2
Surfactant compounds in accordance with the second aspect of the present invention

| | Y | Z | Synthesised and characterized |
|---|---|---|---|
| 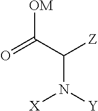 | H H H H R′ or X R′ or X R′ or X | H R′ (CH₂)ₙA (CH₂)ₙCO₂M H R′ (CH₂)ₙA | bCₙGLYM/bCₙ(O)GLYM; ebCₙGLY_{M}/ebCₙ(O)GLY_{M} R′ = CH₃: bCₙALAM/bCₙ(O)ALAM; ebCₙALAM/ebCₙ(O)ALA_{M} n = 0: bCₙp-ABA n = 2, A=OH, Cl: bCₙmEA, bCₙmEC n = 1: bCₙASP_{M}/bCₙ(O)ASP_{M}, ebCₙASP_{M}/ebCₙ(O)ASP_{M}; n = 2: bCₙGLU_{M}/bCₙ(O)GLUM; R′ = CH₃: bCₙSAR_{M}/bCₙ(O)SAR_{M}; ebCₙSAR_{M}/ebCₙ(O)SAR_{M} |
| | R′ or X | (CH₂)ₙCO₂M | n = 1: bCₙASP_{M}/bCₙ(O)ASP_{M}, ebCₙASP_{M}/ebCₙ(O)ASP_{M} |
| | (CH₂)ₙA | H | |
| | (CH₂)ₙA | R′ | |
| | (CH₂)ₙA | (CH₂)ₙA | |
| | (CH₂)ₙA | (CH₂)ₙCO₂M | |
| | (CH₂)ₙCO₂M | H | n = 1: bCₙIDA_{M}/bCₙ(O)IDA_{M}; ebCₙIDA_{M}/ebCₙ(O)IDA_{M} |
| | (CH₂)ₙCO₂M | R′ | |
| | (CH₂)ₙCO₂M | (CH₂)ₙA | |
| | (CH₂)ₙCO₂M | (CH₂)ₙCO₂M | |

X = COR or CH₂R
R = R′CH(CH₂)₂R′ or R′CCHCH₂R′
R′ = alkyl (CₙH₂ₙ₊₁; n = 1, 2, . . .)
n = 0, 1, 2, . . .
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, Cl, Br, I)
M = H, R′, Li, Na, K, Rb, Cs, Er, Gd
bCₙ = R(CH₂)₂CHRCH₂—;
bCₙ(O) = R(CH₂)₂CHRC(═O)—
beCn = R(CH₂)₂CHRCH₂—;
beCn(O) = R(CH₂)₂CHRC(═O)—
ALA = alanine;
ASP = aspartic acid;
GLU = glutamic acid;
GLY = glycine;
IDA = iminodiacetic acid;
SAR = sarcosine.

The surfactant compounds in accordance with the third aspect of the present invention, as synthesized from the above synthesis route, are shown below in Table 3.

TABLE 3
Surfactant compounds in accordance with the third and fourth
aspect of the present invention

| | | Synthesised and |
|---|---|---|
| | Z | characterized |
| 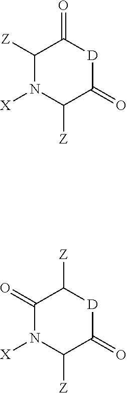 | H R′(CH₂)ₙA (CH₂)ₙCO₂M | D = O: bCₙMPD₂₆ |

X = COR or CH₂R
R = R′CH(CH₂)₂R′ or R′CCHCH₂R′
R′ = alkyl (CₙH₂ₙ₊₁; n = 1, 2, . . .)
D = O, N(CH₂)ₙA, N(C₆H₄)ₙA
n = 0, 1, 2, . . .
A = OH, OR′, SH, SR′, NH₂, NHR′, NR′₂, CONH₂, CONHR′, CONR′₂, aromatic groups (e.g. C₆H₅, C₆H₄OH, C₆H₄COOH, etc.), heteroaromatic groups, halogens (F, CI, Br, I)
M = H, R′, Li, Na, K, Rb, Cs, Er, Gd
MPD₂₆ = morpholine-2,6-dione

The surfactant compounds in accordance with the first, second, third and fourth aspects of the present invention demonstrated the following surfactant properties as shown in Table 4.

TABLE 4
Surfactant Properties

| | Surfactant properties | | | | |
|---|---|---|---|---|---|
| | CMC | [C₂₀] | Π_{CMC} | CMC | γ_{CMC} |
| Surfactant | (mmol/L) | (mmol/L) | (mN/m) | (mg/L) | (mN/m) |
| bC₁₂IDA_{Na} | 99.014 | 2.089 | 40.823 | 34357.84 | 30.71 |
| bC₁₆IDA_{Na} | 3.846 | 0.192 | 42.918 | 1542.20 | 28.17 |
| bC₁₈IDA_{Na} | 0.711 | 0.016 | 45.400 | 305.03 | 27.37 |
| bC₂₀IDA_{Na} | 0.142 | 0.005 | 42.715 | 64.83 | 28.89 |
| bC₁₂ASP_{Na} | 3.412 | 0.169 | 47.128 | 1177.18 | 26.27 |
| bC₁₆ASP_{Na} | 5.132 | 0.043 | 46.031 | 2068.13 | 25.73 |
| bC₁₈ASP_{Na} | 0.826 | 0.013 | 46.309 | 354.17 | 25.33 |
| bC₂₀ASP_{Na} | 0.017 | 0.001 | 46.727 | 7.86 | 25.20 |
| bC₁₈(O)ASP_{Na} | 10.776 | 0.561 | 40.354 | 4776.51 | 31.21 |
| bC₁₈(O)GLU_{Na} | 10.987 | 0.469 | 37.875 | 5020.94 | 33.64 |
| bC₁₈(O)ALA_{Na} | 1.310 | 0.092 | 43.852 | 493.77 | 30.26 |
| bC₁₈(O)GLY_{Na} | 1.005 | 0.095 | 45.360 | 364.77 | 28.39 |
| bC₁₂(O)ALA_{Na} | 43.974 | 0.405 | 42.473 | 12884.36 | 27.21 |
| bC₁₂(O)GLY_{Na} | 54.575 | 0.095 | 39.553 | 15226.32 | 31.55 |
| bC₁₂(O)GLU_{Na} | 41.317 | 4.941 | 39.107 | 15411.31 | 30.22 |
| bC₁₂(O)IDA_{Na} | 30.115 | 0.741 | 39.400 | 11232.84 | 30.06 |
| bC₁₆(O)IDA_{Na} | 30.697 | 0.106 | 41.853 | 13168.92 | 29.66 |
| bC₁₆(O)ALA_{Na} | 5.967 | 0.004 | 43.452 | 2082.42 | 28.06 |
| bC₁₆(O)ASP_{Na} | 10.796 | 0.344 | 42.807 | 4480.24 | 28.44 |
| bC₁₆(O)GLU_{Na} | 24.787 | 0.332 | 41.594 | 10633.55 | 29.71 |
| bC₁₆(O)GLY_{Na} | 4.544 | 0.180 | 42.828 | 1522.09 | 28.68 |

CMC = Critical micelle concentration
[C₂₀] = Surfactant concentration when surface tension has reduced by 20 mN/m (at ca. 52 mN/m)
Π_{CMC} = Surface excess at CMC
γ_{CMC} = Surface tension at CMC

From the above results, it can be seen that the surfactant compounds according to the first and second aspects of the invention have been synthesised which demonstrate superior surfactant properties in comparison to known surfactants.

The description is presented by way of example only in the cause of providing what is believed to be the most useful and readily understood description of the principles and conceptual aspects of the invention. In this regard, no attempt is made to show structural details of the invention and/or the equipment utilized therein in more detail than is necessary for a fundamental understanding of the invention.

1-12. (canceled)

13. A surfactant compound having a chemical structure selected from: 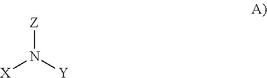
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; and (CH₂)ₙA (n=integer; A=SH, SR′, NH₂, NHR′, NR′₂, CONH₂, CONHR′, CONR′₂, or a halogen); and
(iii) Z is selected from the group consisting of H; R′; and (CH₂)ₙA (n=integer; A=SH, SR′, NH₂, NHR′, NR′₂, CONH₂, CONHR′, CONR′₂, or a halogen); 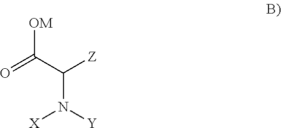
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 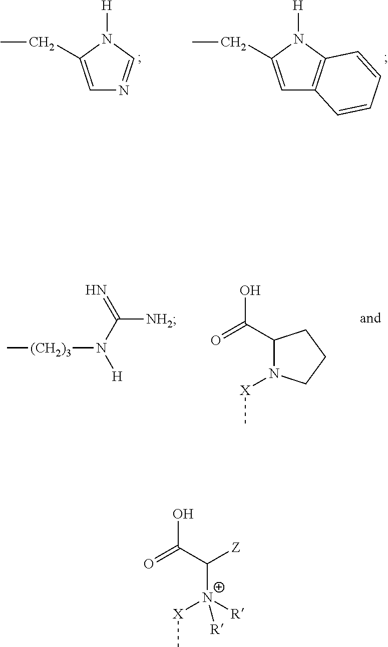
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine; 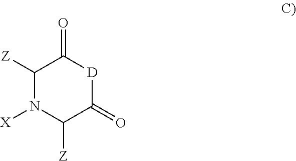
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd); and 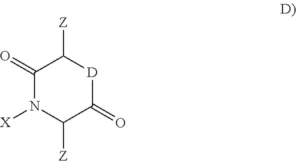
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

14. The surfactant compound according to claim 13, having the following chemical structure: 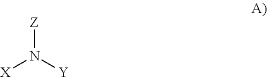
wherein:
(i) X is either COR or CH₂R, wherein R is either R′CH(CH₂)₂R′ or R′CCHCH₂R′, and wherein R′ is CH₂ₙ₊₁ (n=integer);
(ii) Y is selected from the group consisting of H; R′; X; and (CH₂)ₙA (n=integer; A=SH, SR′, NH₂, NHR′, NR′₂, CONH₂, CONHR′, CONR′₂, or a halogen); and
(iii) Z is selected from the group consisting of H; R′; and (CH₂)ₙA (n=integer; A=SH, SR′, NH₂, NHR′, NR′₂, CONH₂, CONHR′, CONR′₂, or a halogen).

15. The surfactant compound according to claim 13, having the following chemical structure: 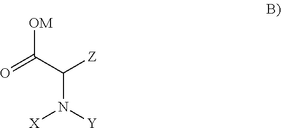
wherein:
(i) X is either COR or CH₂R, wherein R is either R′CH(CH₂)₂R′ or R′CCHCH₂R′, and wherein R′ is CₙH₂ₙ₊₁ (n=integer);
(ii) Y is selected from the group consisting of H; R′; X; (CH₂)ₙCO₂M (n=integer; M=H, R′, Li, Na, K, Rb, Cs, Fr, Er, or Gd); (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and CH₂CH(CH₃)O(CH₂CH₂O)ₙH (n=integer); and
(iii) Z is selected from the group consisting of H; R′; (CH₂)ₙA (n=integer; A=OH, OR′, SH, SR′, NH₂, NHR′, NR′₂, CONH₂, CONHR′, CONR′₂, an aromatic group, a heteroaromatic group, or a halogen); (CH₂)ₙCO₂M (n=integer; M=H, R′, Li, Na, K, Rb, Cs, Fr, Er, or Gd); 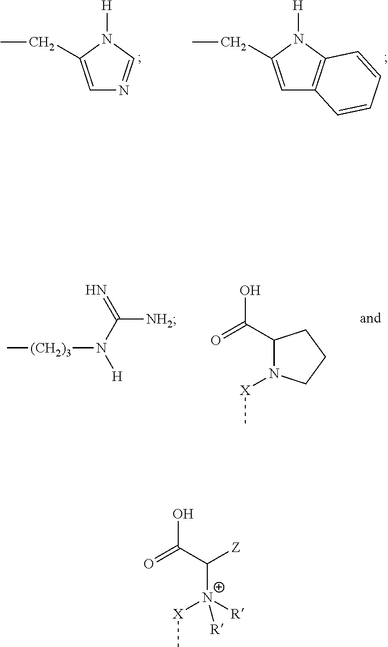
wherein A=OH (SER); SH (CYS); CH₂SMe (MET); COOH (ASP); CH₂COOH (GLU); (CH₂)₃NH₂ (LYS); CONH₂ (ASP); CH₂CONH₂ (GLN); CH(OH)Me (THR); and
wherein ALA=alanine; ASP=aspartic acid; GLU=glutamic acid; GLY=glycine; IDA=iminodiacetic acid; VAL=valine; LEU=leucine; ILE=isoleucine; PHE=phenylalanine; SER=serine; CYS=cysteine; MET=methionine; LYS=lysine; GLN=glutamine; THR=threonine.

16. The surfactant compound according to claim 13, having the following chemical structure: 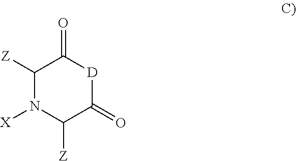
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

17. The surfactant compound according to claim 13, having the following chemical structure: 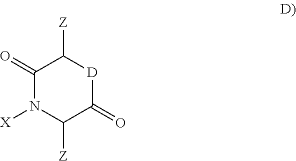
wherein:
(i) X is either COR or CH₂R, wherein R is either R′CH(CH₂)₂R′ or R′CCHCH₂R′, and wherein R′ is CₙH₂ₙ₊₁ (n=integer);
(ii) D is selected from the group consisting of O; N(CH₂)ₙA and N(C₆H₄)ₙA (n=integer; A=OH, OR′, SH, SR′, NH₂, NHR′, NR′₂, CONH₂, CONHR′, CONR′₂, an aromatic group, a heteroaromatic group or a halogen); and
(iii) Z is selected from the group consisting of H; R′; N(CH₂)ₙA (n=integer; A=OH, OR′, SH, SR′, NH₂, NHR′, NR′₂, CONH₂, CONHR′, CONR′₂, an aromatic group, a heteroaromatic group, or a halogen); and (CH₂)ₙCO₂M (n=integer; M=H, R′, Li, Na, K, Rb, Cs, Fr, Er, or Gd).

18. The surfactant compound according to claim 14, wherein surface tension at critical micelle concentration (CMC) is from 27.37 mN/m to 30.71 mN/m.

19. The surfactant compound according to claim 15, wherein surface tension at critical micelle concentration (CMC) is from 25.20 mN/m to 33.64 mN/m.

20. The surfactant compound according to claim 14, wherein X is CH₂R wherein R is R′CH(CH₂)₂R′ and R′ is CₙH₂ₙ₊₁ (n=4, 6, 7 or 8); Y is CH₂CO₂M (M=Na); and Z is CH₂CO₂M (M=Na).

21. The surfactant compound according to claim 14, wherein X is COR wherein R is R′CH(CH₂)₂R′ and R is CₙH₂ₙ₊₁ (n=4 or 6); Y is CH₂CO₂M (M=Na); and Z is CH₂CO₂M (M=Na).

22. The surfactant compound according to claim 14, wherein X is CH₂R, wherein R is R′CH(CH₂)₂R′ and R′ is CₙH₂ₙ₊₁ (n=7 or 8); Y is CH₂CO₂M (M=Na); and Z is CH₂CO₂M (M=Na).

23. The surfactant compound according to claim 15, wherein X is CH₂R, wherein R is R′CH(CH₂)₂R′ and R′ is CₙH₂ₙ₊₁ (n=4, 6, 7 or 8); Y is H; and Z is CH₂CO₂M (M=Na).

24. The surfactant compound according to claim 15, wherein X is COR wherein R is R′CH(CH₂)₂R′ and R′ is CₙH₂ₙ₊₁ (n=4, 6 or 7); Y is H; and Z is H, CH₃, or (CH₂)ₙCO₂M (n=1 or 2; M=Na).

25. The surfactant compound according to claim 15, wherein X is CH₂R wherein R is R′CH(CH₂)₂R′ and R′ is CₙH₂ₙ₊₁ (n=9); Y is H; and Z is CH₂CO₂M (M=Na).
